(19) United States
(12) Patent Application Publication
(10) Pub. No.: US 2009/0018525 A1
(43) Pub. Date: Jan. 15, 2009

(54) Tapered catheter devices

(75) Inventors: Sarah E. Waite, Cory, IN (US); Thomas A. Osborne, Bloomington, IN (US); Jason C. Urbanski, Ellettsville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/827,962

(22) Filed: Jul. 13, 2007

(51) Int. Cl. A61M 25/098

(52) U.S. Cl. 604/508; 604/104

(57) ABSTRACT

Catheters, catheter systems, and methods of using catheters to deliver therapy are disclosed. Advantages include increased torqueability and control with catheters, having a soft distal tip. Catheter systems including a sheath and catheter are disclosed wherein the catheter fits the sheath with a dilator-like fit.

### BACKGROUND

The present invention relates to medical devices and more particularly, to catheters and catheter systems.

Catheters are used in a variety of medical procedures. Commonly, catheters are used to provide drainage, inject fluids, place medical devices, and to otherwise provide access to locations with the body. Whether within the vascular system, or in other systems, the insertion of a catheter carries with it certain risks due to the often delicate nature of the anatomy through which the catheter is inserted. Examples of catheters include guide catheters, delivery catheters angioplasty catheters, stent delivery devices, angiographic catheters, and neurocatheters.

Angiography is one example of a medical procedure involving catheters. Angiography is conducted to determine the presence of narrowing or blockage in the arteries, determine the risk of future stroke, or to determine the need for further treatment such as angioplasty, stenting or bypass surgery.

One type of angiography is carotid angiography. During carotid angiography, a catheter is inserted into a blood vessel, typically at the arm or leg. The catheter may be inserted through a valve to prevent blood reflux, air aspiration, and unintended movement of the catheter. With the help of a medical imaging machine, the catheter is guided through the arteries until the catheter is in the vicinity of the carotid artery. Contrast dye may be used to aid in the navigation of the catheter through the anatomy or at the location of the carotid artery.

In addition, catheters typically are used in medical procedures such as angioplasty and stenting. Angioplasty and stenting require delivery of therapy through a sheath rather than open vascular access. Successful sheath delivery facilitates angioplasty and stenting procedures. Successful sheath delivery depends upon an ability to assess the internal anatomy, catheterize the vascular branches, perform remote access guidewire-catheter manipulation, and place a sheath.

The variety of catheter uses and locations for use require a number of catheter shapes and sizes. Catheters for specific applications often have necessary or preferred characteristics. For example, a catheter must be an appropriate length in order to allow the target within the body to be reached. In addition, catheters are commonly preferred to have a certain pushability and torqueability to aid in the maneuvering of the catheter through the various twists and turns of the anatomy through which the catheter is being inserted. When catheters are used with guidewires or multiple catheters are used, inside and outside diameters become important to consider.

One type of catheter has a radiographic tip. Catheters with radiographic tips are particularly useful in angiography. Angiographic selective catheters are known to have a variety of conformations. For example, they can be entirely straight, have an angled tip, have a pigtail conformation, or they can have a portion of the distal end bent back towards the straight section. One problem associated with catheters is that it is possible for the catheter tip to puncture the sides of the vessels in which it is inserted. Another problem associated with catheters is that the shaft may be too rigid due to the diameter of the catheter or the materials used. For this and a number of other reasons, it is apparent to the inventors that catheters and catheter systems with improved characteristics are needed.

### SUMMARY

A catheter system is provided with a sheath and a catheter. The sheath and catheter have a dilator-like fit between the outer diameter of the catheter and the inner diameter of the sheath. Tapered devices are also provided. Methods of performing intravascular procedures are also provided.

### BRIEF DESCRIPTION OF SEVERAL VIEWS OF THE DRAWINGS

The invention may be more fully understood by reading the following description in conjunction with the drawings, in which:

FIG. 1A is a side view of a catheter system;

FIG. 1B is a cross-sectional view of the catheter of the catheter system of FIG. 1A;

FIG. 1C is a partial cross-sectional view of the catheter of FIG. 1B and a sheath from the catheter system of FIG. 1A;

FIG. 2A is a cross-sectional view of a catheter;

FIG. 2B is a cross-sectional view of a catheter;

FIG. 3 is a cross-sectional view of an asymmetric catheter; and

FIG. 4 is a flow chart of a method of performing an intravascular procedure with a catheter system.

### DETAILED DESCRIPTION

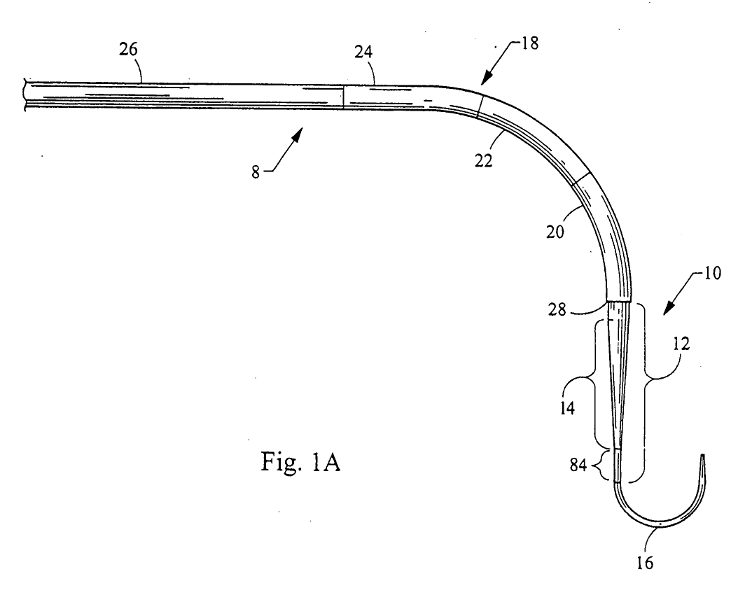

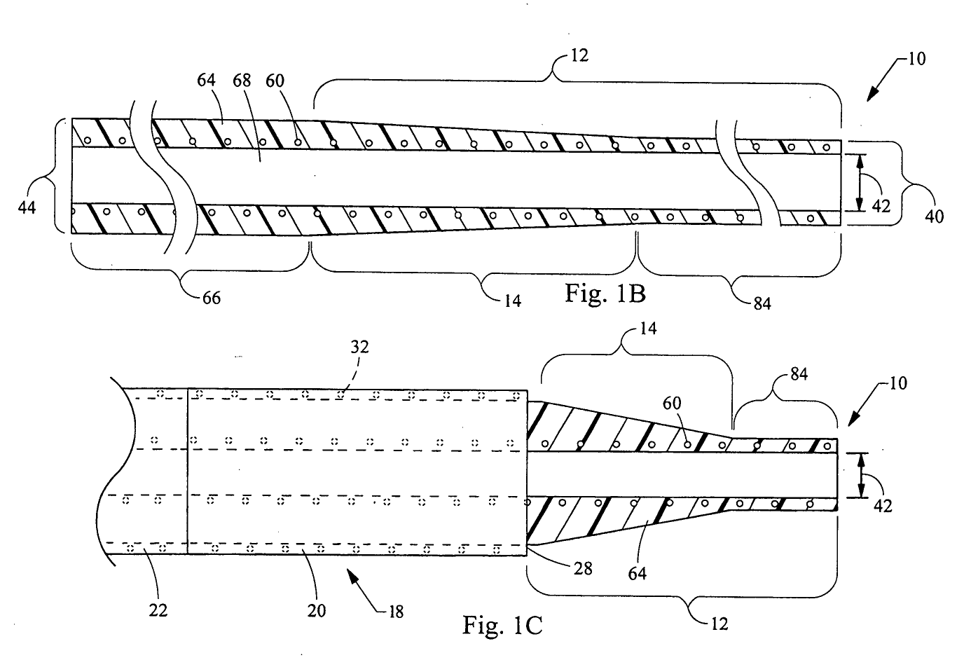

Referring to the drawings, and in particular FIG. 1A, FIG. 1A shows one embodiment of a catheter system 8. The catheter system 8 comprises a sheath 18 and a catheter 10 located coaxial and internal to the sheath 18.

The sheath 18 includes four regions of distinct composition and distinct flexibility. The regions may comprise different materials, or optionally may be comprised of the same materials in different arrangements or compositions. The sheath 18 may also consist of as few as one region, but preferably comprises at least four regions. Proximally, the sheath includes a first sheath region 26 that is relatively stiff and has the least flexibility of the four sheath regions shown. The second sheath region 24 is distal to the first sheath region 26 and is more flexible than the first sheath region 26. The third sheath region 22 is distal to the second sheath region 24. The fourth sheath region 20 is distal to the third sheath region 22. The distal end 28 of the catheter may be rounded inside and out for presenting an atraumatic end surface. At the most distal end 28 of the sheath 18, the sheath 18 forms a substantially close fit, or “dilator-like fit,” with the catheter 10 when the catheter is in an ordinary coaxially position as shown in FIG. 1A. The four sheath regions 26, 24, 22, 20 have a substantially constant outer diameter and a substantially constant inner diameter. The sheath 18 preferably has an outside diameter of between about 4 French and about 7 French, an inner diameter of between about 0.0595 inches and about 0.100 inches, and a wall thickness of between about 0.006 inches and about 0.125 inches. Preferably, the sheath has an outside diameter of about 6 French, an inner diameter of about 0.087 inches, and an outside diameter of about 0.090 inches.

The distal region 12 of the catheter 10 is shown in FIG. 1A. The distal region 12 includes an intermediate tapering region 14. The proximal region of the catheter 10 has a substantially constant outer diameter. The tapering region 14 preferably tapers from an initial diameter to a final diameter by about 1.5 to 2.5 French sizes. For example, the catheter 10 may taper from an initial diameter of about 5.5 French to about 4.0 French. The catheter 10 may also taper from an initial diameter of about 5.5 French to about 3.0 French. The tapering region 14 is preferably between about 2 to about 7 centimeters in length. More preferably, the tapering region 14 is between about 3 to about 4 centimeters long. Distal to the tapering region 14 is a region of constant diameter 84. A radiopaque tip 16 is connected to the region of constant diameter 84.

Referring now to FIG. 1B, the distal end and a portion of the proximal end 66 of the catheter 10 from FIG. 1A is shown. The catheter 10 has a region of constant diameter 84 and a distal region 12 which includes the tapering region 14. The catheter 10 includes a polymer shaft 64, a first structural braid 60, and an inner lumen 68. The first structural braid 60 is disposed within the polymer shaft 64. As partially shown, the first structural braid 60 extends within the polymer shaft 64 through the entire length of the catheter 10. The catheter 10 has an initial diameter 44 in a region proximal to the tapering region 14. The catheter has a distal outer diameter 40 that is smaller than the initial diameter 44. The catheter 10 has a constant inner diameter 42 throughout the length of the catheter. Not shown in this figure is the radiopaque tip 16 which is attached to the distal end of the catheter 10.

FIG. 1C is a close-up cross-sectional view of the distal end of the sheath 18 and catheter 10 from FIG. 1A. As shown, the catheter 10 includes a first structural braid 60 disposed within the polymer shaft 64. The catheter 10 includes a tapering region 14 located within the distal region 12 and a region of constant diameter 84. The distal region 20 of the sheath 18 is shown and the second-most distal region 22 is partially shown. The sheath 18 includes a second structural support element 32 disposed within the wall of the sheath 18.

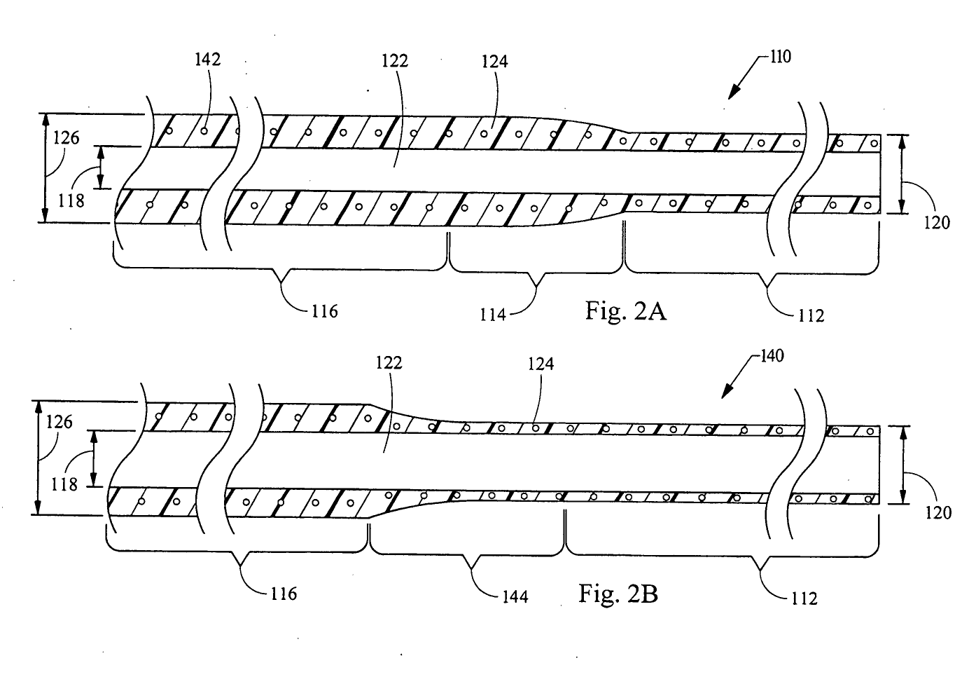

Referring now to FIG. 2A, FIG. 2A shows a catheter 110 with a proximal region 116, a tapering region 114, and a distal region 112. The distal region 112 has a substantially constant outer diameter 120. The catheter 110 includes a polymer shaft 124 with a structural braid 142 disposed within the polymer shaft 124. In this embodiment, the tapering region 114 is convex. The catheter has a proximal outer diameter 126 and a distal outer diameter 120 that is smaller than the proximal outer diameter 126. The inner lumen 122 has a constant diameter 118 throughout. Not shown in FIGS. 2A and 2B are radiopaque tips, such as tip 16 shown in FIG. 1A, which may be attached to the distal end of the catheter 110.

Referring now to FIG. 2B, FIG. 2B shows a catheter 140 with a proximal region 116, a tapering region 144, and a distal region 112. The distal region 112 has a substantially constant outer diameter 120. The catheter 140 includes a polymer shaft 124 with a structural braid disposed within the polymer shaft 124. In this embodiment, the tapering region 144 is concave. The catheter 140 has a proximal outer diameter 126 and a distal outer diameter 120 that is smaller than the proximal outer diameter 126. The inner lumen 122 has a constant diameter 118 throughout.

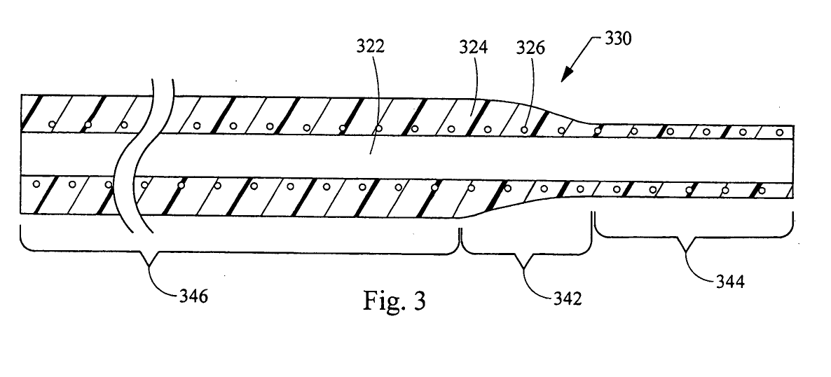

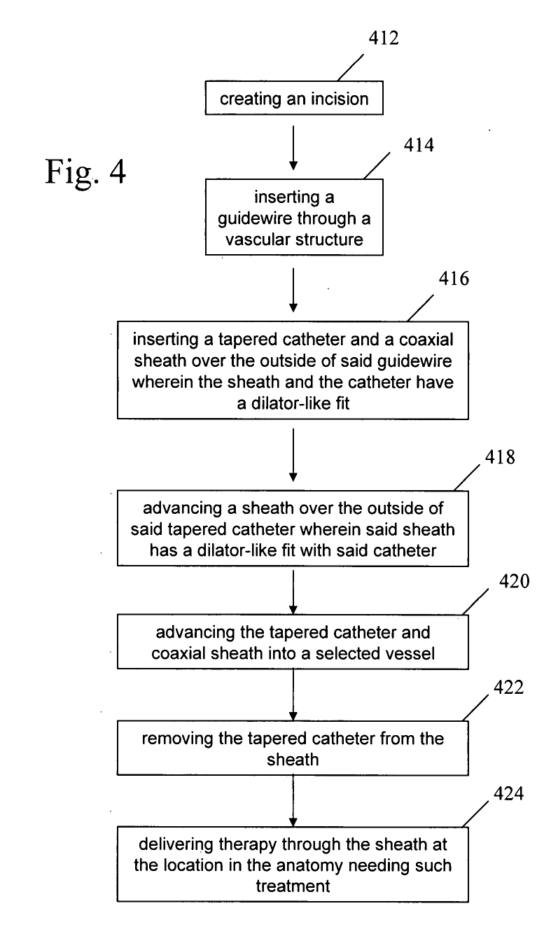

Referring now to FIG. 3, FIG. 3 shows an asymmetric catheter 330 with a proximal region 346, a tapering region 342, and a distal region 344. The catheter includes a polymer shaft 324 with a structural braid 326 disposed within the polymer shaft 324. In this embodiment, the tapering region 342 is asymmetric.

The inner diameter of the catheter is preferably in the range of between about 0.039 inches and 0.047 inches. Preferably, the catheters described herein include a radiopaque tip attached to the distal end of the catheter. The radiopaque tip 16 (shown in FIG. 1A) may be attached by any means commonly known in the art. For example, the radiopaque tip 16 may be attached by mechanical means or thermal processing. The radiopaque tip 16 may have any number of shapes known in the art. For example, the radiopaque tip 16 may be straight, hooked, or pigtail. According to the invention, the radiopaque tip 16 has an outer diameter at the proximal end that is 1.5 to 2.5 Fr. smaller than the diameter of the proximal region of the catheter shaft. Preferably, the radiopaque tip 16 is curved when external to the sheath 16, but may be straightened while the catheter 10, 110, 140, 330 is inserted in the sheath. The radiopaque tip 16 may be curved to aid in the selection of particular vessels during an intraluminal procedure. Optionally, the tip 16 may be pigtail shaped which may prevent trauma to vessel walls.

In one embodiment, the region of constant diameter 84, 112, 344 is preferably between about 2 centimeters and about 5 centimeters. More preferably, the region of constant diameter 84, 112, 344 is between about 3 to about 4 centimeters. The length of the region of constant diameter 84, 112, 344 may be selected based upon the method that is used to attach the radiopaque tip 16 to the catheter 10, 110, 140, 330. In one embodiment, the radiopaque tip 16 is thermally bonded to the catheter 10, 110, 140, 330 with a process that requires a region of constant diameter 84, 112, 344 of at least about 3 centimeters.

Preferably the catheter includes a catheter and a catheter sheath which is designed to be advanced over the catheter. The catheter may be designed to be advanced over a wire guide into the desired location within a body vessel. The sheath may be designed to be highly torqueable and steerable in order to provide access to vessels. The sheath is preferably large enough for typical medical procedures to be performed through the inner lumen of the sheath. For example, a stent may be advanced through the sheath after the catheter is withdrawn. Sheath embodiments may also be used to introduce devices to the accessed vessels, such as embolic protection devices, stent delivery systems, and additional catheters.

Sheath regions preferably have distinct differences in durometer values. A proximal region of the sheath has a first durometer. Moving distally, the next region of the sheath has a second durometer. The sheath may include a third, fourth, and fifth region having a third, fourth, and fifth durometer, and so on. The successive regions of the sheath preferably are of decreasing durometer. A sheath with regions of decreasing durometer offers the advantage of allowing a sheath to have a relatively stiff shaft while allowing for a relatively soft tip.

In certain procedures, a sheath with a very soft tip is desired. For example, in carotid stenting, a stent is placed in the carotid artery. The carotid artery is a particularly delicate part of anatomy. Devices may cause damage the walls of the carotid artery if the tip of the catheter or sheath is not an appropriate durometer or flexibility. In applications requiring a very soft tip to work in a particularly sensitive portion of the anatomy, the durometer of the shaft may be significantly greater than the durometer of the tip. The decrease in durometer may be achieved by one or more than one sheath sections.

A problem associated with sheaths with connected regions that have significantly different flexibilities is that the sheath may kink. In particular, when a region of relatively low flexibility is connected to a region of high flexibility, the likelihood of kinking may be increased. Kinking may lead to negative effects. For example, kinking may prevent a sheath from being advanced or retracted. Moving a kinked sheath may also damage the surrounding vessel walls. Accordingly, it is desirable to include regions of increasing flexibility which may prevent kinking.

The sheath may have a helical braid for structural support. The helical braid may comprise nylon, stainless steel wire, or any other suitable stiffening material. The coils of the braid are preferably equally spaced throughout the length of the sheath. Optionally, the coils of the braid are not evenly spaced. For example, the coils of the braid may be located further apart in the distal end of the sheath than the proximal part of the sheath. The increased space between coils may lead to an increase in flexibility towards the distal end. This may be desirable and may achieve a similar effect to regions of distinct compositions having distinct flexibility.

Catheter system embodiments may utilize an oversized catheter with respect to a sheath. It is common in the art to refer to the size of a sheath by the size of catheter that will be inserted. For example, a 7 French catheter will typically be used with a 7 French sheath. However, embodiments of this invention preferably utilize a catheter which is larger than normal. For example, a 7.5 French catheter may be used with a 7.0 French sheath.

One advantage of this type of configuration is that the catheter may fit to the sheath like a dilator typically fit to a sheath; i.e. there is a substantially close fit between the catheter and sheath and the fit is “dilator-like.” By dilator-like fit, what is meant is that the fit between the outside surface of the catheter and inside surface of the sheath is between about 0.0015 inches and about 0.002 inches. There may also be essentially no gap between the outer sheath and tapered catheter. As a result of the close fit between the catheter and sheath, negative effects such as backflow may be reduced or prevented. An additional benefit of the close fit between the catheter and sheath is that the fit may provide more control over the catheter by reducing the amount of movement permitted between the catheter and sheath. Yet another benefit may be a reduction of the so-called “bulldozer” effect. The “bulldozer” effect refers to the ability of certain catheter systems to permit the formation of a “shovel” due to the tolerance between the sheath and the catheter. As catheter systems are passed through the anatomy, the “shovel” may knock off plaque or thrombus. When a catheter and sheath have a substantially close fit, the “bulldozer” effect may be reduced.

In embodiments of the invention in which the catheter is larger than typically used, a sheath with a very soft distal end is advantageous. During periods in which the catheter is inserted through a sheath and beyond the distal end of the sheath, the substantially close fit between the catheter and sheath may provide an increase in control over manipulations of the sheath and catheter.

The outer diameter of the catheter is preferably in the range of about 2.5 French to about 9.5 French, or about 0.033 inches to about 0.1245 inches. More preferably, the outside diameter of the catheter is in the range of about 4.5 French to about 7.5 French, or 0.058 about inches to 0.0985 about inches.

Preferably, the catheter includes a structural braid or coil. The structural braid or coil may be embedded in the polymer shaft of the catheter and may have a constant diameter. The structural braid or coil may be disposed within the outer layer of the catheter. It may be incorporated in the outer layer in a process having multiple steps. For example, a braid or coil may be placed on a first layer of material. A second layer may then be added on top of the structural braid or coil, and the layers may be treated in a way to produce one seamless outer layer to form the polymer shaft. Other methods of providing structural support may also be included in the catheter.

Preferably, the structural braid or coil is stainless steel within a nylon polymer. In addition, the coil or braid preferably extends substantially throughout the length of the catheter from the proximal end to the distal end. Preferably, the coils or braid are spaced uniformly throughout the length of the catheter. Also, structural members in certain embodiments of the invention may be spaced in changing increments.

Catheter and sheath embodiments may have a lubricious polymer coating on the interior surface, outer surface, or both surfaces. Preferably, the interior surface is not coated with a lubricious polymer coating, but instead is comprised of a lubricious material, such as PTFE. A lubricious polymer coating may be particularly suited for manipulating medical devices in close proximity to each other and to vessel walls. Preferably, the lubricious coating is a hydrophilic coating that becomes slippery when a fluid such as water is applied.

A method of performing an intravascular procedure with a catheter system is described below. The catheter may be inserted into a body in ways commonly known in the art. For example, an incision may be made 412 at an access point, such as the femoral artery, and a guidewire is inserted through the vascular structure 414. The guidewire may be inserted with a tapered catheter or with multiple catheters external to the guidewire. A dilator may be employed at the point of insertion to facilitate insertion of the catheter.

A sheath and a coaxial tapered catheter are advanced with a coaxial tapered catheter 418. The sheath and coaxial tapered catheter have a dilator-like fit. The catheter is advanced through the anatomy and into a selected vessel 420. The catheter and sheath may be advanced in multiple stages. For example, the guidewire may be initially advanced a certain distance and a tapered catheter may be advanced over the guidewire followed by additional advancements of the guidewire. The guidewire may also be advanced in one stage to the desired location. The sheath and tapered catheter may be advanced concurrently. Multiple catheters may be inserted with the sheath. When the sheath is in the desired location, the catheter with the dilator-like fit with the sheath is retracted from the sheath 422. The sheath is maintained in the desired location during the retraction. Therapy is delivered through the sheath at the location needing treatment 424. For example, a stent may be deployed thought the sheath. For further example, an imaging device may be use in conjunction with the catheter system.

While preferred embodiments of the invention have been described, it should be understood that the invention is not so limited, and modifications may be made without departing from the invention. The scope of the invention is defined by the appended claims, and all devices and methods that come within the meaning of the claims, either literally or by equivalence, are intended to be embraced therein. Furthermore, the advantages described above are not necessarily the only advantages of the invention, and it is not necessarily expected that all of the described advantages will be achieved with all embodiments of the invention.

1-17. (canceled)

18. A method of using a tapered catheter for performing an intravascular procedure comprising the steps of:
creating an incision;
inserting a guidewire through a vascular structure;
inserting a tapered catheter and a coaxial sheath over the outside of said guidewire;
wherein said sheath has a dilator-like fit with said catheter;
advancing the tapered catheter and coaxial sheath into a selected vessel;
removing the tapered catheter from the sheath;
delivering therapy through the sheath at the location in the anatomy needing such treatment.

19. The method of claim 18, wherein the therapy delivered comprises delivering a stent through said sheath.

20. The method of claim 19, wherein the location needing such treatment is the carotid artery.
